(19) United States
(12) Patent Application Publication
(10) Pub. No.: US 2006/0241305 A1
(43) Pub. Date: Oct. 26, 2006

(54) Amorphous form of losartan potassium

(76) Inventors: Yatendra Kumar, Haryana (IN); Tarun Kant Sharma, Bihar (IN); Prosenjit Bose, Haryana (IN)

Correspondence Address: RANBAXY INC., 600 COLLEGE ROAD EAST, SUITE 2100, PRINCETON, NJ, 08540, US

(21) Appl. No.: 10/532,887

(22) PCT Filed: Oct. 31, 2003
(86) PCT No.: PCT/IB03/04873

§ 371 Date: May 24, 2006

(30) Foreign Application Priority Data
Oct. 31, 2002 (IN) 1095/DEL/2002

(51) Int. Cl. C07D 403/02; A61K 31/4178

(52) U.S. Cl. 548/253; 514/381

(57) ABSTRACT

This invention relates to an amorphous form of losartan potassium. The invention also relates to processes for preparing amorphous losartan potassium and pharmaceutical compositions that include the amorphous losartan potassium.

### FIELD OF THE INVENTION

The field of the invention relates to an amorphous form of losartan potassium. The invention also relates to processes for preparing amorphous losartan potassium and pharmaceutical compositions that include the amorphous losartan potassium.

### BACKGROUND OF THE INVENTION

Chemically, losartan potassium is 2-butyl-4-chloro-1-[[2′-(1H-tetrazol-5-yl)[1,1′-biphenyl]-4-yl]methyl]-1H-imida zole-5-methanol and has structural Formula I 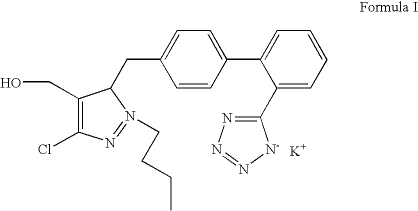
It is disclosed in U.S. Pat. No. 5,138,069. Losartan potassium is a substituted imidazole useful as an angiotensin II receptor antagonist. It is known for treating hypertension and congestive heart failure.

U.S. Pat. No. 5,608,075 discloses novel crystalline forms of losartan potassium and describes two novel polymorphic forms, differing from one another in respect of their physical properties, stability, and spectral data. They are designated Form I and Form II. It is known that different morphs of biologically active compounds may have different absorption profile in vivo and consequently different pharmacokinetic profile.

### SUMMARY OF THE INVENTION

In one general aspect there is provided an amorphous form of losartan potassium.

The amorphous form of losartan potassium may have the infrared spectrum of FIG. 1 and the X-ray diffraction pattern of FIG. 2.

In another general aspect there is provided a pharmaceutical composition that includes a therapeutically effective amount of an amorphous form of losartan potassium; and one or more pharmaceutically acceptable carriers, excipients or diluents.

In another general aspect there is provided a process for the preparation of the amorphous form of losartan potassium. The process includes preparing a solution of losartan potassium in one or more solvents; and recovering the losartan potassium in the amorphous form from the solution thereof by the removal of the solvent.

The solvent may be one or more of lower alkanol, ketone, chlorinated solvent, water or mixtures thereof. The lower alkanol may include one or more of primary, secondary and tertiary alcohol having from one to six carbon atoms. The lower alkanol may include one or more of methanol, ethanol, denatured spirit, n-propanol, isopropanol, n-butanol, isobutanol, and t-butanol. In particular, the lower alkanol may include one or more of methanol, ethanol, and denatured spirit.

The ketone may include one or more of acetone, 2-butanone, and 4-methylpentan-2-one.

The chlorinated solvent may include one or more of chloroform and dichloromethane.

Removing the solvent may include one or more of distillation, distillation under vacuum, evaporation, spray drying, freeze drying, filtration, filtration under vacuum, decantation and centrifugation.

The losartan potassium in an amorphous form may be recovered from the solution by spray drying. Alternatively, the losartan potassium in an amorphous form may be recovered from the solution by freeze-drying. The process may include further forming of the product so obtained into a finished dosage form.

The amorphous form of losartan potassium can also be recovered from the solution by adding a suitable non-solvent resulting in the precipitation of the amorphous form and removing the solvent there from by filtration, decantation or centrifugation. The non-solvent may be selected from a group of organic solvents in which losartan potassium is insoluble or poorly soluble or practically insoluble or partially soluble and is known to a person of ordinary skills in the art.

The process may include further drying of the product obtained from the solution.

The process may produce the amorphous form of the losartan potassium having the infrared spectrum of FIG. 1 and the X-ray diffraction pattern of FIG. 2.

The details of one or more embodiments of the inventions are set forth in the description below. Other features, objects and advantages of the inventions will be apparent from the description and claims.

### DESCRIPTION OF THE DRAWINGS

FIG. 1 is an infrared spectrum in KBr of amorphous form of losartan potassium.

FIG. 2 is X-ray powder diffraction pattern of amorphous form of losartan potassium.

FIG. 3 is an infrared spectrum showing peaks characteristic of crystalline form I and form II of losartan potassium from 1150 cm⁻¹ to 600 cm⁻¹ obtained per U.S. Pat. No. 5,608,075: (A) Form I and (B) Form II.

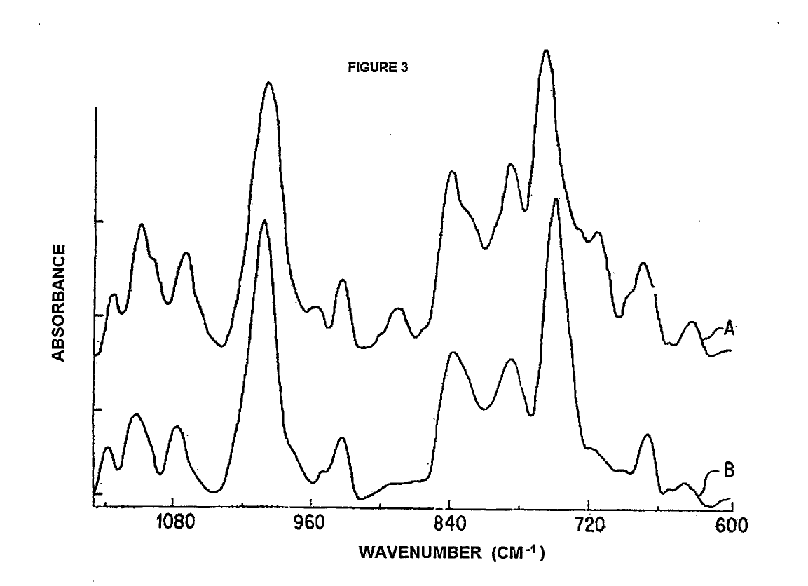

FIG. 4 is an infrared spectrum showing peaks characteristic of crystalline form I and form II of losartan potassium from 1800 cm⁻¹ to 1150 cm⁻¹ obtained per U.S. Pat. No. 5,608,075: (A) Form I and (B) Form II.

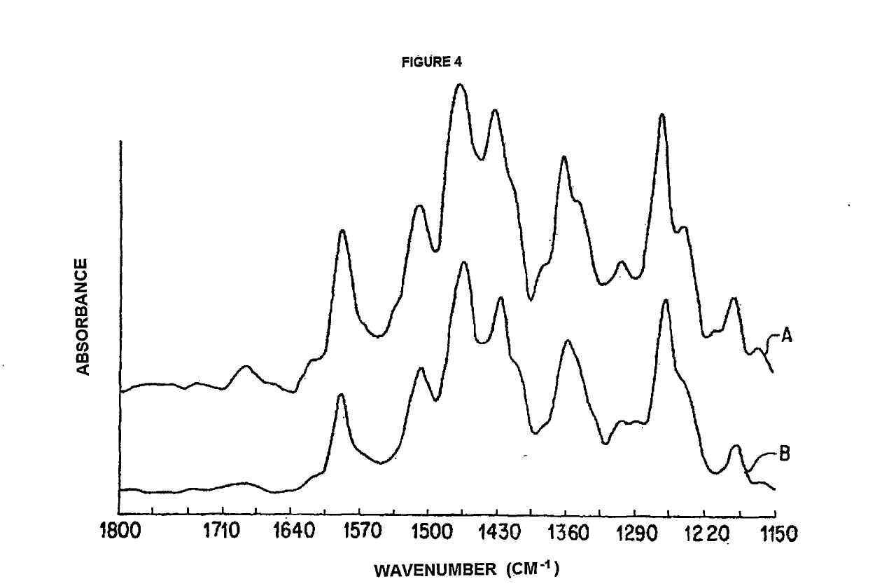

FIG. 5 is an X-ray diffraction pattern characteristic of crystalline forms I and II of losartan potassium obtained per U.S. Pat. No. 5,608,075: (A) Form I and (B) Form II.

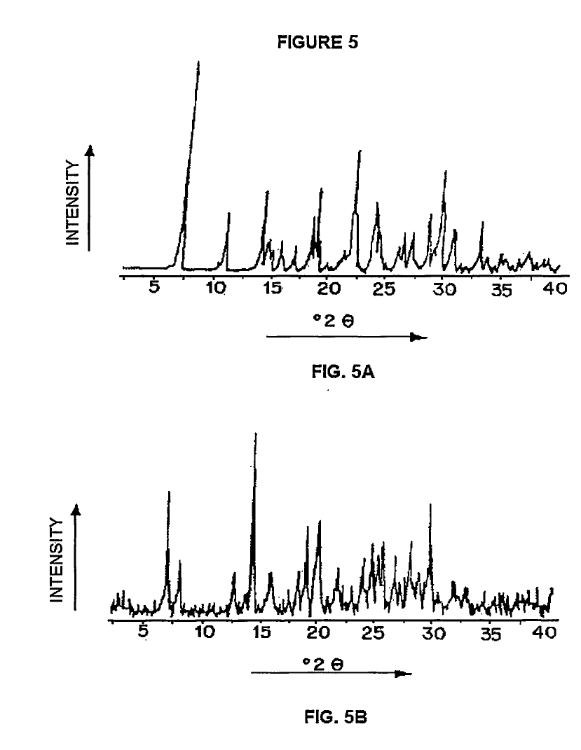

### DETAILED DESCRIPTION OF THE INVENTION

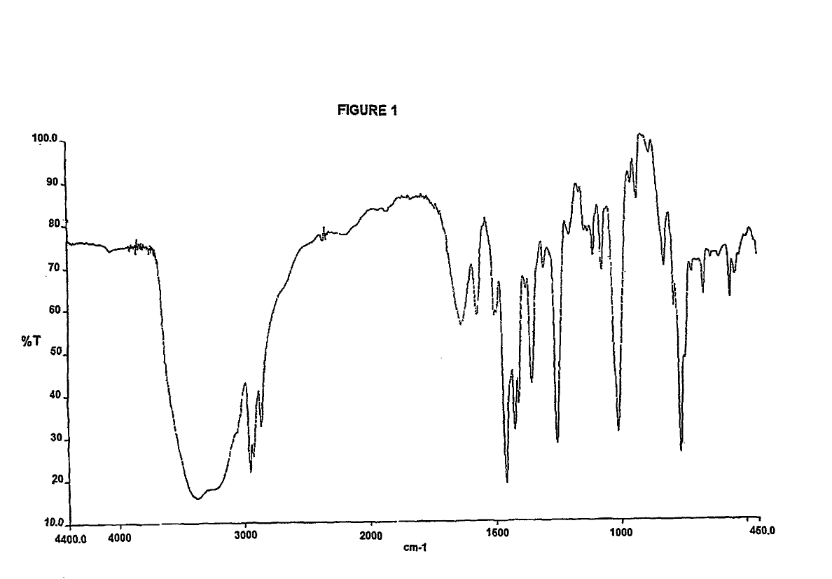

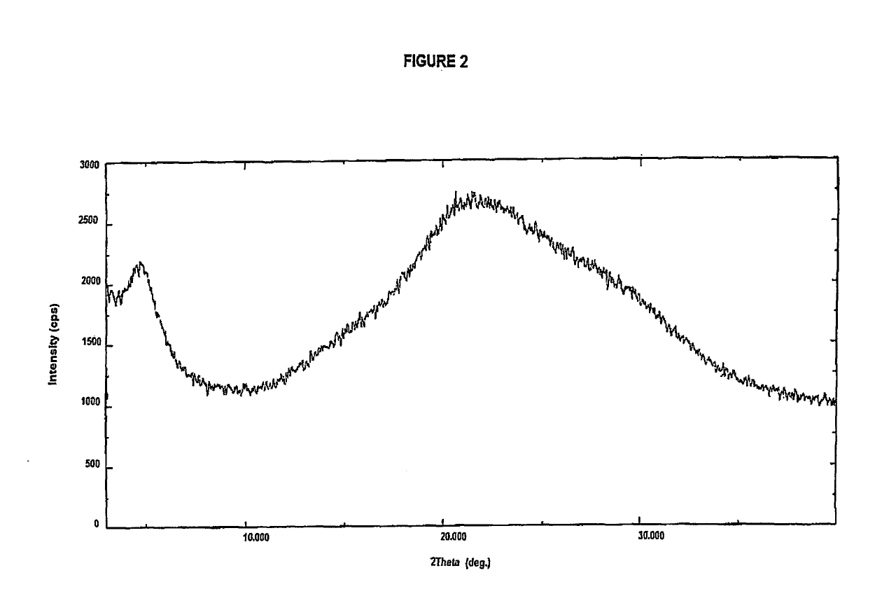

The inventors have found a new form of losartan, the amorphous form and, in particular, the amorphous losartan potassium. The new form is characterized by its infrared spectrum and X-ray powder diffraction pattern as shown in FIGS. 1 and 2, respectively. The inventors also have developed a process for the preparation of the amorphous form of losartan potassium, by recovering the amorphous losartan potassium from a solution thereof in a suitable solvent by spray drying. The inventors also have developed pharmaceutical compositions that contain the amorphous form of the losartan potassium, in admixture with one or more solid or liquid pharmaceutical diluents, carriers, and/or excipients.

In general, the solution of losartan potassium may be obtained by dissolving a crystalline losartan potassium in a suitable solvent. Alternatively, such a solution may be obtained directly from a reaction in which losartan potassium is formed. The solvent may be removed from the solution by a technique which includes, for example, distillation, distillation under vacuum, evaporation, spray drying, freeze drying, filtration, decantation, and centrifugation.

In one aspect, losartan potassium in amorphous form is recovered from the solution using a spray drying technique. A Mini-Spray Dryer (Model: Buchi 190, Switzerland) can be used. The Buchi 190 Mini-Spray Dryer operates on the principle of nozzle spraying in a parallel flow, i.e., the sprayed product and the drying gas flow in the same direction. The drying gas can be air or inert gases such as nitrogen, argon and carbon dioxide.

In another aspect, losartan potassium in amorphous form can be recovered from the solution using a freeze drying technique. A freeze dryer (Model: Virtis Genesis SQ Freeze Dryer) can be used in this technique. The Virtis Genesis SQ Freeze Dryer operates on the principle of lyophilization, i.e., a process of stabilizing initially wet materials (aqueous solution or suspensions) by freezing them, then subliming the ice while simultaneously desorbing some of the bound moisture (primary drying). Following removal of the ice, desorption may be continued (secondary drying). This process may be carried out under vacuum.

The term “suitable solvent” includes any solvent or solvent mixture in which losartan potassium, is soluble, including, for example, lower alkanol, ketones, chlorinated solvents, water and mixtures thereof. Examples of alkanol include those primary, secondary and tertiary alcohols having from one to six carbon atoms. Suitable lower alkanol solvents include methanol, ethanol, denatured spirit, n-propanol, isopropanol, n-butanol, isobutanol and t-butanol. Examples of ketones include solvents such as acetone, 2-butanone, and 4-methylpentan-2-one. A suitable chlorinated solvent includes one or more of dichloromethane, dichloroethane and chloroform. Mixtures of all of these solvents are also contemplated.

If crystalline losartan potassium is used as a starting material it may be in the form of any of the various polymorphic forms known in the prior art including solvates, hydrates, anhydrous or any other polymorphic forms of losartan potassium. A solution of losartan potassium obtained in situ during the preparation process may be used as such for spray drying.

The spray drying may be accomplished using a spray dryer which operates on the principle of nozzle spraying in a parallel flow, i.e., the sprayed product and the drying gas flow in the same direction. The drying gas can be air or one or more inert gases such as nitrogen, argon, and carbon dioxide. Moreover, the product obtained may be further or additionally dried to achieve the desired moisture values. For example, the product may be further or additionally dried in a tray drier, dried under vacuum and/or in a Fluid Bed Dryer.

The resulting amorphous form of losartan potassium may be formulated into ordinary dosage forms such as, for example, tablets, capsules, pills, solutions, etc. In these cases, the medicaments can be prepared by conventional methods with conventional pharmaceutical excipients.

The compositions include dosage forms suitable for oral, buccal, rectal, and parenteral (including subcutaneous, intramuscular, and ophthahnic) administration. The oral dosage forms may include solid dosage forms, like powder, tablets, capsules, suppositories, sachets, troches and lozenges as well as liquid suspensions, emulsions, pastes and elixirs. Parenteral dosage forms may include intravenous infusions, sterile solutions for intramuscular, subcutaneous or intravenous administration, dry powders to be reconstituted with sterile water for parenteral administration, and the like.

The present invention is further illustrated by the following example which is provided merely to be exemplary of the invention and is not intended to limit the scope of the invention. Although the example is directed to amorphous form of losartan potassium, the principles described in this example can be applied to other salts of amorphous losartan.

Preparation of Amorphous Form of Losartan Potassium

### EXAMPLE

A suspension was made from crystalline losartan potassium (10 g) in methanol (300 ml) at ambient temperature. The resulting solution was slowly heated to 45-47° C. for 30 minutes to get a clear solution which was subjected to spray drying in a Mini Spray Dryer model Buchi-190) at a temperature of 67-68° C. using nitrogen gas. The losartan potassium in an amorphous form was collected. It was further dried at 45-50° C. for 8 hours under vacuum to yield amorphous losartan potassium.

X-ray powder diffraction pattern (FIG. 2) showed a plain halo, which demonstrates the amorphous nature of the product. Infrared spectrum in KBr (FIG. 1) is different than one obtained for crystalline form of losartan potassium.

While the present invention has been described in terms of its specific embodiments, certain modifications and equivalents will be apparent to those skilled in the art and are intended to be included within the scope of the present invention.

We claim:

1. An amorphous form of losartan potassium.

2. The amorphous form of losartan potassium of claim 1, wherein the losartan potassium has the infrared spectrum of FIG. 1.

3. The amorphous form of losartan potassium of claim 1, wherein the losartan potassium has the X-ray diffraction pattern of FIG. 2.

4. A pharmaceutical composition comprising:
a therapeutically effective amount of an amorphous form of losartan potassium;
and one or more pharmaceutically acceptable carriers, excipients or diluents.

5. The pharmaceutical composition of claim 1, wherein the losartan potassium has the infrared spectrum of FIG. 1.

6. The pharmaceutical composition of claim 1, wherein the losartan potassium has the X-ray diffraction pattern of FIG. 2.

7. A process for the preparation of the amorphous form of losartan potassium, the process comprising:
preparing a solution of losartan potassium in one or more solvents; and
recovering the losartan potassium in the amorphous form from the solution thereof by the removal of the solvent.

8. The process of claim 7, wherein the solvent comprises one or more of lower alkanol, ketone, chlorinated solvent, water, or mixtures thereof.

9. The process of claim 8, wherein the lower alkanol comprises one or more of primary, secondary and tertiary alcohol having from one to six carbon atoms.

10. The process of claim 8, wherein the lower alkanol comprises one or more of methanol, ethanol, denatured spirit, n-propanol, isopropanol, n-butanol, isobutanol, and t-butanol.

11. The process of claim 8, wherein the lower alkanol comprises one or more of methanol, ethanol, and denatured spirit.

12. The process of claim 8, wherein the ketone comprises one or more of acetone, 2-butanone, and 4-methylpentan-2-one.

13. The process of claim 8, wherein the chlorinated solvent comprises one or more of chloroform, dichloromethane, and dichloroethane.

14. The process of claim 7, wherein removing the solvent comprises one or more of distillation, distillation under vacuum, evaporation, spray drying, freeze drying, filtration, decantation, and centrifugation.

15. The process of claim 7, wherein the losartan potassium in an amorphous form is recovered from the solution by spray drying.

16. The process of claim 7, wherein the losartan potassium in an amorphous form is recovered from the solution by freeze-drying.

17. The process of claim 7, wherein the losartan potassium in an amorphous form is recovered from the solution by filtration.

18. The process of claim 7, further comprising additional drying of the product obtained.

19. The process of claim 7, further comprising forming the product obtained into a finished dosage form.

20. The process of claim 7, wherein the losartan potassium has the infrared spectrum of FIG. 1.

21. The process of claim 7, wherein the losartan potassium has the X-ray diffraction pattern of FIG. 2.
